(19) United States
(12) Patent Application Publication
(10) Pub. No.: US 2014/0120067 A1
(43) Pub. Date: May 1, 2014

(54) MUSCLE STEM CELL THERAPY FOR TREATMENT OF DYSPHAGIA

(71) Applicant: Stacey L. Halum, Indianapolis, IN (US)

(72) Inventor: Stacey L. Halum, Indianapolis, IN (US)

(73) Assignee: Indiana University Research and Technology Corporation, Indianapolis, IN (US)

(21) Appl. No.: 14/068,820

(22) Filed: Oct. 31, 2013

Related U.S. Application Data
(60) Provisional application No. 61/720,542, filed on Oct. 31, 2012.

(51) Int. Cl. A61K 35/34

(52) U.S. Cl. CPC A61K 35/34
USPC 424/93.7

(57) ABSTRACT

Described herein is a method for the treatment of dysphagia, comprising administering a therapeutically effective dose of an isolated muscle-derived stem cell population into a muscle involved in swallowing, such as tongue, pharynx, palate, and strap muscles.

### CROSS-REFERENCE TO RELATED APPLICATIONS

This application claims priority to U.S. Provisional Application Ser. No. 61/720,542 filed Oct. 31, 2012, which is incorporated herein by reference in its entirety.

### FIELD OF THE INVENTION

The invention relates generally to stem cells and medicine, and more particularly to muscle-derived stem cells and neurogenic dyshpagia.

### BACKGROUND

Dysphagias are swallowing disorders that are a major problem in the United States, affecting over 10,000 individuals each year. Swallowing is a highly complex process involving both nerve and muscle function. Difficulty in swallowing can occur as consequence of disease to either the organs and muscles involved in swallowing, or more commonly to the central nervous system controlling swallowing, called neurogenic dysphagia. Neurogenic dysphagia with base of tongue weakness often results after head and neck cancer therapy (surgery and/or chemoradiation), but can also be caused by neurological disorders such as stroke, multiple sclerosis, Parkinson's disease, muscular dystrophies, and head injuries. Currently, other than swallowing exercises to try to strengthen the tongue muscles, there are no beneficial treatment options to improve or restore swallowing in these patients. Thus, most of these patients are rendered feeding (g-tube) dependent. As such, a need for a more effective beneficial treatment option is needed for patients with dysphagia.

### BRIEF SUMMARY

In one aspect described herein is a method for treating dysphagia, comprising administering to a subject in need thereof a therapeutically effective dose of an isolated muscle-derived stem cell population into a muscle used for swallowing.

In some embodiments the muscle used for swallowing is selected from the group consisting of a tongue muscle, a pharynx muscle, a palate muscle, and a strap muscle.

In some embodiments at least about 10⁴ to about 10⁸ muscle-derived stem cells are administered to the subject. In some embodiments, the muscle-derived stem cell population to be administered is generated from sternocleidomastoid muscle. In some embodiments the muscle-derived stem cell population is autogenic, allogenic, or xenogenic.

In some embodiments the subject to be treated is suffering from neurogenic dysphagia. In some embodiments the subject to be treated is human.

In some embodiments the treatment method also includes obtaining and culturing the isolated muscle-derived stem cell population prior to its administration to the subject in need thereof. In some embodiments the treatment results in lingual strength at least 10% greater to about 30% greater than lingual strength in the subject prior to the treatment.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, wherein:

FIG. 1 shows placement of the nerve cuff electrode (A and B) and the tongue tensometer and high resolution manometry set up (C); and

FIG. 2 shows histological staining of sheep tongue pre- and post partial nerve injury and bungarotoxin beta3-tubulin staining; and

FIG. 3 shows myofiber diameter of two individual sheep tongues pre- and post-partial nerve injury; and

FIG. 4 shows autologous MdSCs from two sheep, cultured and transduced to express EGFP (+CD56 and +EGFP via FACS); and

FIG. 5 shows cross sectional tongue muscle fiber specimen (12 um) from Sheep 1 imaged at ×20 magnification in fluorescent (A) and brightfield (B) modes indicating the presence of GFP+ myofibers (arrows in B) and the presence of fused MdSC at two-months post injection in certain areas of the tongue; and

FIG. 6 shows detection of GFP marker by immunohistochemistry with GFP+ myofibers (bottom central set of arrows) representing the areas where injected GFP+MdSCs have fused with innate myofibers; note that GFP+ myofibers appear larger in diameter than GFP− (upper right set of arrows) myofibers; and

FIG. 7 shows bungarotoxin-beta3 tubulin stains of tongue specimens (12 um) at baseline (A and C) and post-denervation injury (B and D), indicating a reduction in nerve to motor end plate (indicated by arrows) contact in both animals.

FIG. 8 shows force of tongue contraction in sheep 1 pre- and post-muscle stem cell implantation; and

FIG. 9 shows mean muscle fiber diameter for GFP+ (striped bars) are larger than GFP− (white bars) muscle fibers in both animals post-MSC, suggesting attenuation of muscle fiber atrophy where MSCs fused; and

FIG. 10 shows representative High Resolution Manometery output from Sheep 1 Pre-MSC (A) and post-MSC injection (B), indicating an increase in base of tongue (BOT) and upper esophageal sphincter (UES) pressure two-months post-MSC injection (B); and

FIG. 11 shows mean muscle fiber diameter for GFP+ myofibers increased across time points while GFP− myofiber diameter decrease indicating formation of new muscle fibers and atrophy of myofibers post-denervation injury respectively.

### DETAILED DESCRIPTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements.

Likewise, many modifications and other embodiments of the present invention set forth herein will come to mind to one of skill in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

Overview

The tongue is a critical organ for the finely controlled processes of articulation and deglutition. Tongue muscle mass and functional movement may be significantly impaired in patients with stroke, degenerative neurologic disease, and head and neck cancer. The paralyzed, weakened or fibrotic tongue can result in dysarthria and dysphagia with devastating sequelae. These include: malnutrition and dehydration (1-3), profound dysphagia with an inability to eat by mouth, communication disability with a reliance on augmentative communication devices (4), aspiration causing pneumonia and death (5-7) and compromised quality of life, mental well-being, social functioning, anxiety, and depression (8-11). Current therapies for lingual dysfunction such as oral motor exercises are suboptimal for restoring muscle mass and functional dynamic motion of the tongue. In addition, they place high demands on individuals who must perform daily exercises while in a compromised state.

The utilization of autologous muscle-derived stem cell (MdSC) therapy to treat the damaged tongue is an appealing treatment strategy. MdSCs have the ability to restore both muscular deficits and dynamic function (12, 13). The tongue is technically easy to inject and is highly accessible. In addition, the injection of a patient's own, autologous MdSCs is safe. MdSC therapy has been shown to be effective in other organ systems. Myoblast injection has improved myocardial function in individuals with end-stage coronary artery disease with significant improvements in cardiac ejection fraction noted across a number of clinical trials (14, 15). In a rodent model of unilateral recurrent laryngeal nerve injury, Halum et al. (12-16) demonstrated that autologous MdSCs survive and attenuate atrophy of denervated myofibers in the larynx.

Although autologous MdSC therapy has promise for the treatment of the denervated, weakened or fibrotic tongue, little work has been done in this area. To date, no study has investigated the effects of autologous MdSC therapy on lingual strength in a denervated large animal tongue model, or other muscles involved in swallowing. As such, the current disclosure describes a use of MdSCs in a manufacture of a medicament for the treatment of dysphagia in a subject, comprising administering a therapeutically effective dose of an isolated muscle-derived stem cell population into a muscle involved in swallowing.

Dysphagia

Dysphagia is a condition typified by a decreased ability to swallow. The normal swallowing of a human (or mammal) involves three distinct phases that are interdependent and well coordinated: (i) the oral, (ii) the pharyngeal, and (iii) the esophageal phases. In the oral phase, which is under voluntary control, food that has been chewed and mixed with saliva is formed into a bolus for delivery by voluntary tongue movements to the back of the mouth, into the pharynx. The pharyngeal phase is involuntary and is triggered by food/liquid bolus passing through the faucial pillars into the pharynx. Contraction of the three constrictors of the pharynx propel the bolus towards the upper esophageal sphincter. Simultaneously, the soft palate closes the nasopharynx. The larynx moves upwards to prevent food or liquid passing into the airway, which is aided by the backward tilt of the epiglottis and closure of the vocal folds. The esophageal phase is also involuntary and starts with the relaxation of the upper esophageal sphincter followed by peristalsis, which pushes the bolus down to the stomach.

Oral dysphagia is a very serious condition and is generally not treatable with medication. Oral dysphagia affects individuals of all ages, but is more prevalent in older individuals. Worldwide, oral dysphagia affects approximately 22 million people over the age of 50. When dysphagia is caused by disease or impairment of the nervous system, this is termed “neurogenic dysphagia”. Dyphagia is often a consequence of an acute event, such as a stroke, brain injury, or surgery for oral or throat cancer. In addition, radiotherapy and chemotherapy may weaken the muscles and degrade the nerves associated with the physiology and nervous innervation of the swallow reflex. It is also common for individuals with progressive neuromuscular diseases, such as Parkinson's Disease, to experience increasing difficulty in swallowing initiation. Representative causes of dysphagia include those associated neurological illnesses (brainstem tumors, head trauma, stroke, cerebral palsy, Guillain-Barre syndrome, Huntington's disease, multiple sclerosis, polio, post-polio syndrome, metabolic encephalopathies, amyotrophic lateral sclerosis, Parkinson's disease, dementia), infectious illnesses (diphtheria, botulism, Lyme disease, syphilis, mucositis [herpetic, cytomegalovirus, Candida, etc.]), autoimmune illnesses (lupus, scleroderma, Sjogren's syndrome), metabolic illnesses (amyloidosis, cushing's syndrome, thyrotoxicosis, Wilson's disease), myopathic illnesses (connective tissue disease, dermatomyositis, myasthenia gravis, myotonic dystrophy, oculopharyngeal dystrophy, polymyositis, sarcoidosis, paraneoplastic syndromes, inflammatory myopathy), iatrogenic illnesses (medication side effects, e.g., chemotherapy, neuroleptics, etc., post surgical muscular or neurogenic, radiation therapy, corrosive (e.g. pill injury, intentional), Tardive Dyskinesia, a chronic disorder of the nervous system characterized by involuntary jerky movements of the face, tongue, jaws, trunk, and limbs, usually developing as a late side effect of prolonged treatment with antipsychotic drugs, and structural illnesses such as cricopharyngeal bar, Zenker's diverticulum, cervical webs, oropharyngeal tumors, osteophytes and skeletal abnormalities, congenital (e.g. cleft palate, diverticulae, pouches, etc.).

Dysphagia is not generally diagnosed although the disease has major consequences on patient health and healthcare costs. Individuals with more severe dysphagia generally experience a sensation of impaired passage of food from the mouth to the stomach, occurring immediately after swallowing. Among community dwelling individuals, perceived symptoms may bring patients to see a doctor. Among institutionalized individuals, health care practitioners may observe symptoms or hear comments from the patient or his/her family member suggestive of swallowing impairment and recommend the patient be evaluated by a specialist. As the general awareness of swallowing impairments is low among front-line practitioners, dysphagia often goes undiagnosed and untreated. Yet, through referral to a swallowing specialist (e.g., speech language pathologist), a patient can be clinically evaluated and dysphagia diagnosis can be determined.

The general awareness of swallowing impairments is low among front-line practitioners. Many people (especially those who are elderly) suffer with undiagnosed and untreated swallowing impairments. One reason is that front-line community care practitioners (e.g., general practitioners/geriatricians, home care nurses, physical therapists, etc.) do not typically screen for the condition. If they are aware of the severity of swallowing impairments, they commonly do not use an evidence-based method of screening. Furthermore, office-based assessment of dysphagia rarely occurs.

Severity of dysphagia may vary from: (i) minimal (perceived) difficulty in safely swallowing foods and liquids, (ii) an inability to swallow without significant risk for aspiration or choking, and (iii) a complete inability to swallow. Many people with swallowing impairment do not seek medical care when symptoms are mild or unrecognized. For example, “silent aspiration,” a common condition among elderly, refers to the aspiration of the oropharyngeal contents during sleep. People may compensate for less-severe swallowing impairments by self-limiting the diet. The aging process itself, coupled with chronic diseases such as hypertension or osteoarthritis, predisposes elderly to (subclinical) dysphagia that may go undiagnosed and untreated until a clinical complication such as pneumonia, dehydration, malnutrition (and related complications) occurs. Yet, the differential diagnosis of ‘aspiration pneumonia’ is not necessarily indicated as a result of current care practices.

The economic costs of dysphagia are associated with hospitalization, re-hospitalization, loss of reimbursement due to pay for performance (“P4P”), infections, rehabilitation, loss of work time, clinic visits, use of pharmaceuticals, labor, care taker time, childcare costs, quality of life, increased need for skilled care. Dysphagia and aspiration impact quality of life, morbidity and mortality. Twelve-month mortality is high (45%) among individuals in institutional care who have dysphagia and aspiration. The economic burden of the clinical consequences arising from lack of diagnosis and early management of dysphagia are significant.

Dysphagia can also occur as a result of surgical resection of oral cancer, a procedure called a glossectomy. Depending on the site and extension of the tumor, the possible tongue resections are: marginal glossectomy (resection of one-quarter of the tongue), hemiglossectomy (resection of half of the tongue along the midline), hemiglossomandibulectomy (resection of half of the tongue and a portion of the mandible), and near total glossectomy. After marginal or hemiglossectomy, dyphagias are usually temporary and are mainly related to clumsiness of tongue movement and difficulties in triggering the swallowing reflex.

Muscle-Derived Stem Cells.

As used herein, “muscle-derived stem cells, “muscle stem cells”, “muscle-derived progenitor cells”, “muscle progenitor cells”, “MdSCs”, “MSCs” and the singular is meant a self-renewing mononucleate cell that produces as progeny mononucleate myoblasts, which are committed to form multinucleate myofibers via intercellular fusion. Encompassed by the disclosure are MdSCs that produce skeletal muscle In an additional embodiment, the muscle stem cells are of skeletal muscle origin. In another embodiment, the muscle stem cells are derived from sternocleidomastoid muscle.

“Myogenic” cells as described herein are those cells that are related to the origin of muscle cells or fibers. Various molecular markers are known to be specific for the middle and late stages of myogenic differentiation. For example, in C2C12 cells, myosin and MRF4 mark the late stages of myogenesis and are largely restricted to myotubes, whereas myogenin and nestin mark the middle stages of myogenesis and are found in all myotubes and in many committed myoblasts.

Myoblasts are mononucleated muscle cells, are uniquely different from other cells in the body in a number of ways: 1) myoblasts naturally differentiate to form muscle tubules capable of muscle contraction, 2) when myoblasts fuse to form myotubes, these cells become post mitotic (stop dividing) with maturation, thus allowing control of the number and amount of myoblasts per injection, and 3) as myotubes, the cells express large amounts of protein which is produced in the cells due to multinucleation. Myoblasts transferred into mature muscle tissue will proliferate and differentiate into mature muscle fibers. This process involves the fusion of mononucleated myogenic cells (myoblasts) to form a multinucleated syncytium (myofiber or myotube).

As used herein, “subject” or “patient” means an individual having symptoms of, or at risk for, dysphagia, neurogenic dysphagia, or other malignancy. A patient may be human or non-human and may include, for example, animal strains or species used as “model systems” for research purposes, such a sheep model as described herein. Likewise, patient may include either adults or juveniles (e.g., children). Moreover, patient may mean any living organism, preferably a mammal (e.g., human or non-human) that may benefit from the administration of compositions contemplated herein.

As used herein, “isolated” when used in reference to cells, means a single cell of interest, or population of cells of interest, at least partially isolated or purified from other cell types or other cellular material with which it naturally occurs in the tissue of origin (e.g., adipose tissue). A sample of MdSCs is “isolated” when it is at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% and, in certain cases, at least about 99% free of cells other than cells of interest. Purity can be measured by any appropriate method, for example, by fluorescence-activated cell sorting. The isolated muscle-derived stem cells may include support cells. In a particular embodiment, the isolated sample of muscle stem cells comprises satellite cells and myoblasts.

As used herein, the terms “administering,” “introducing,” “delivering,” “placement” and “transplanting” are used interchangeably herein and refer to the placement of the MdSCs of the disclosure into a subject by a method or route which results in at least partial localization of the MdSC at a desired site. The MdSCs can be administered by any appropriate route which results in delivery to a desired location in the subject where at least a portion of the cells or components of the cells remain viable. The period of viability of the cells after administration to a subject can be as short as a few hours, e.g., twenty-four hours, to a few days, to as long as several years.

As used herein, “therapeutically effective dose” refers to an amount of MdSCs that are sufficient to bring about a beneficial or desired clinical effect. Said dose could be administered in one or more administrations. However, the precise determination of what would be considered an effective dose may be based on factors individual to each patient, including, but not limited to, the patient's age, size, type or extent of disease, stage of the disease, route of administration of the regenerative cells, the type or extent of supplemental therapy used, ongoing disease process and type of treatment desired (e.g., aggressive vs. conventional treatment). In one embodiment, at least about 10⁴ MdSCs are administered. In another embodiment, at least about 10⁵ MdSCs are administered. In an additional embodiment, at least about 10⁶ MdSCs are administered. In another embodiment, at least about 10⁷ MdSCs are administered. In another embodiment at least about 10⁸ MdSCs are administered.

As used herein, “sample,” means a biological sample from a subject, including, but not limited to, normal tissue samples, diseased tissue samples, biopsies, blood, saliva, feces, semen, tears, and urine. A sample can also be any other source of material obtained from a subject that contains cells, tissues, or fluid of interest. A sample can also be obtained from cell or tissue culture.

As used herein, the term “allogeneic tissue”, “allogeneic cell”, or “allogeneic” refers to a tissue or cell which is isolated from an individual and used in another individual of the same species. The term “xenogeneic tissue”, “xenogeneic cell” or “xenogeneic” refers to a tissue or cell which is isolated from an individual of one species and placed in an individual of another species. The term “autogeneic tissue”, “autogeneic cell”, or “autogenic” refers to a tissue or cell which is isolated from an individual and grafted back into that individual.

Swallowing

Swallowing involves coordination of the sequence of activation and inhibition for more than 30 pairs of muscles in the mouth, pharynx, larynx, and esophagus (Table 1—Russi et. al. Cancer Treatment Reviews, 2012, 38:1033-1049). The term “a muscle involved in swallowing” means any muscle known in the art to be responsible for the action of swallowing that has impaired function. MdSCs can be administered to repair neurogenic damage to any of the muscles involved in swallowing, for example, the tongue, pharynx, palate, strap muscles, and muscles involved in facial expressions.

TABLE 1
Normal swallow phases¹⁵¹

| | Oral | | |
|---|---|---|---|
| Oral proparatory phase | chamber⁴ | Anatomy¹⁵¹ (cranial nerve) | Specific function |
| Teeth, lips, cheeks, tongue, mandible, | Entrance | Lips and orbicularis oris (VII) | Seal lips/mouth |
| and palate grind and manipulate food | valve | | |
| Food mixed with saliva | Floor | Intrinsic tongue | |
| Formulation of a bolus consistency | | longitudinal sup and inf. | Bolus preparation, formation, transport |
| appropriate for safe swallow | | transversis and vertical (XII) | |
| Oral propulsive or transport stage | | Extrinsic tongue: | |
| Lips and cheeks contract | | Hyoglossus (XII) | Down-retraction of the tongue |
| Tongue presses the bolus against the | | Cenioglossus (XII) | Protrude/retract tongue |
| hard palate and soft palate elevates | | Styloglossus (XII) | Raise/retract tongue |
| Bolus is moved backwards by the | | Suprahyoid muscles: | Raise/stabilize hyoid, tongue, mouth floor |
| tongue | | Mylohyoid (V3) | |
| A central groove is formed is the | | Geniohyoid (XII) | Protract/stabilize hyoid |
| tongue for passage of bolus | | Anterior (V3) posterior belly | Raise stabilize hyoid and lower mandible |
| Bolus is moved to the tonsillar pillars, | | digastric (VII) | |
| thus initiating the oral phase of | Lateral wall | Buccinator (VII) | Push food towards teeth |
| swallowing | | Dentition (V2-V3) | Mastication |
| The soft palate moves superior and | Roof | Tensor veli palatinae (V3) | Tense soft polate |
| posterior to close off nasopharynx | | Levator veli palatine (X | Raise soft palate to seal nasopharynx |
| Piston-like action of tongue to propel | | pharyngeal plexus) | |
| food posteriorly | | Palatoglossus (X pharyngeal | Sphincter that seals oral cavity from oropharynx |
| Contraction of mylohyoid muscle | | plexus) | |
| causes this movement | | Uvular (X pharyngeal plexus) | Brace soft polate |
| | Exit valve | Base of tongue | Seal oral cavity and push food in oropharynx |
| | | Palatoglossus (X) pharyngeal | Shincter sealing oral cavity from oropharynx |
| | | plexus | |
| | | Styloglossus (XII) | Raise and retract tongue helping to seal oral cavity |
| | | Palstopharyngeus (X pharyngeal | Raise pharynx and larynx and lower palate sealing oral cavity |
| | | plexus) | |
| | Supportive | Salivary glands: submandibular | Salivation |
| | structures | and sublingual VII; parotid (IX) | |
| | | Mandibula, teeth and and | Stabilize mandible to permit suprahyoid muscles action⁵⁴ |
| | | dentures³⁴ | |
| | | Omohyoid (C1-C2 ansa | Infrahyoid muscles are activated in oral phase and inhibited (but |
| | | cervicallis) | thyrohyoid) during deglutition |
| | | Stemohyoid (C1-C2) | |
| | | Stemohyoid (C1-C2) | |
| Oropharyngeal phase²⁶ | Pharyngeal | Anatomy¹⁵¹(cranial nerve) | Specific function |
| | chamber⁴ | | |
| Retroversion of the epiglott is over the | Entrance | The apposition of the base of the | See exit valve of oral chamber |
| laryngeal vestibule | valve | tongue to the velum and palatus- | |
| Closure of the larynx to the level of | | pharyngeus muscles | |
| the false and true vocal cords | Laryngeal | Epoglotic swinging | Epiglottis swings down to cover laryngeal vestibule |
| The larynx is pulled up and forward | Valves | Thyroepiglottic (recurrent X) | Approach epiglottis to arytenoids |
| by pharynx longitudinal muscles and | | Supraglottic adductors | in continuity with ary-epiglottic m. helps to approach arytenoids |
| pavement of mouth's muscles. | | Ary-epiglottic muscles in folds | to epiglottis and adduct vocal cord |
| Contraction of the PCM | | (recurrent X) | |
| Relaxation of the cricopharyngeal | | Oblique arytenoids (recurrent X) | |
| muscles | | Glottic adductor muscles | Adduct vocal cord |
| Opening of the cricopharyngeal | | Transverse arytenoid | |
| sphincter | | (recurrent X) | |
| by upward and forward movement | | Thyroarytenoid (recurrent X) | |
| of the larynx | | Lateral cricoarytenoid | |
| | | (recurrent X) | |
| Time: duration appropriately 1 s) | | Posterior cricoarytenoid | Open vocal folds |
| | | (recurrent X) | |
| | Rhino- | Tensor veli palatine (V3) | Tense soft palate |
| | pharyngeal | Levator veli palatine (XI | Raise soft palate to seal nasopharynx |
| | valve | pharyngeal plexus | |
| | | Superior PCM (X vagus via | Narrows rhino-pharyngeal volumen sealing it |
| | | pharyngeal plexus) | |
| | Pharyngeal | Longitudinal group of muscles; | Elevates and shortens pharynx |
| | wall | Palatopharingeus (X vagus via | Raise larynx, shorten pharynx |
| | | pharyngeal plexus) | (Sytlopharyngeus also widen pharynx) |
| | | Salpingopharingeus (X vagus via | |
| | | pharyngeal plexus) | |
| | | Stylopharingeus (innervated by | |
| | | IX glossopharyngeus) | |
| | | Stylohyoid (VII) | It is not strictly part of wall (se supportive structure) |
| | | Circular group of muscles: | Peristalsis and bolus transport |
| | | Superior PCM (X vagus via | |
| | | pharyngeal plexus) | |
| | | Middle PCM (X vagus via | |
| | | pharyngeal plexus) | |
| | | Middle PCM (X vagus via | |
| | | pharyngeal plexus) | |
| | | Inferior PCM (X vagus via | |
| | | pharyngeal plexus recurrent and | |
| | | laryngeal nerves) | |
| | Exit valve | Cricopharyngeal sphincter (X) | |
| | Supportive | Mylohyoid) (V1) | Raise and move forward larynx bringing the larynx to a position |
| | structures | Anterior (V3) Posterior belly | under the base of the tongue |
| | | digastric (VII) | |
| | | Geniohyoid (XII) | |
| | | Thyrohyoid (XII) | |
| | | Omohyoid (C1-C2 ansa | Infrahyoid m. are activated in oral phase and inhibited (but |
| | | cervicalis) | thyrohyoid) during deglutition |
| | | Sternohyoid (C1-C2) | |
| | | Sternohyoid (C1-C2) | |
| | | Hyoid bone | The hyoid has mechanical connections to the cranial base, |
| | | Stylohyoid (VII) | mandible, sternum, and thyroid cartilage via the suprahyoid and |
| | | | infrahyoid muscles. With those muscles connections, the hyoid |
| | | | plays an important rule in controlling the movements of the jaw |
| | | | and tongue |
| Oesophageal phase²⁶ | Gesophageal | Anatomy¹⁵¹(cranial nerve) | Specific function |
| | chamber⁴ | | |
| Peristatic contractions of muscular | Entrance | Inferior PCM (X vagus via | Three important factors contribute to the UOES opening: |
| results in movement of the bolus into | valveUOES | pharyngeal plexus recurrent and | (1) Relaxation of the cricopharyngeus muscle; this relaxatiom |
| the stomach | | laryngeal n.) | normally precedes opening of the UOES or arrival of the bolus |
| Time: (duration approximately 3-4 s) | | Cricophsryngeal sphincter | (2) Contraction of the suprahyoid muscles and thyrohyoid |
| | | Oesophagus inlet | muscles. These muscles pull the hyo-laryngeal complex forward, |
| | | muscle(OEM)¹³² | |
| | | Suprahyoid muscles and | opening the sphincter |
| | | thyrohyoid muscles | (3) The pressure of the descending bolus |

Normal swallowing can be broken down into three basic steps or phases. The first step is called the oral phase because it is the process of mechanically chewing the food bolus, working it around with the tongue, and ultimately propelling the food back into the throat. At this point, the pharyngeal phase begins, where the muscles of the throat, or pharynx, work to propel the food bolus toward the esophagus. The muscles of the pharynx are skeletal muscles called the superior, middle and inferior constrictor muscles. During this phase, the soft palate closes against the back wall of the pharynx (throat) to prevent the food bolus from traveling upward toward the nose. The voice box, or larynx, simultaneously closes to keep food from entering the airway. The larynx is also drawn forward and upward, and this helps open the entrance of the esophagus. The esophagus is a tube-like structure that carries food down to the stomach. It has two “gateways,” called the upper esophageal sphincter and the lower esophageal sphincter. The upper esophageal sphincter is located at the entrance of the esophagus. It opens to let food through, and closes after the food bolus passes to help propel the food into the esophagus and to prevent the food from regurgitating back up. The muscle that makes up this sphincter is called the cricopharyngeus muscle. After the food bolus passes through the upper esophageal sphincter, the esophageal phase of swallowing begins. The esophagus is surrounded by an inner layer of muscle fibers (circular layer) that has fibers running circumferentially around the esophagus, and an outer muscle layer (longitudinal layer) that has fibers running parallel to the esophagus. These muscles are under involuntary control, and act to propel the food bolus down to the stomach. Before the food reaches the stomach, it passes one final “gateway” called the lower esophageal sphincter. This sphincter opens to allow passage of the food. This sphincter is also at the level of the diaphragm, with the esophagus lying above the diaphragm and the stomach lying below the diaphragm. Because of its location, the pressure gradient across the diaphragm can influence the function of the lower esophageal sphincter.

### Derivation, Culture, and Transplantation of Muscle-Derived Stem Cells (MdSCs)

Described herein is a method for treating dysphagia, comprising administering to a subject in need thereof a therapeutically effective dose of an isolated muscle-derived stem cell population into a muscle used for swallowing.

MdSCs include stem cells derived from any skeletal muscle, e.g., from the sternocleidomastoid muscle. Such muscle-derived stem cell populations can be obtained, as described herein, from the subject to be treated (i.e., an autogenic cell population), another subject of the same species (i.e., an allogenic cell population), or a subject from a different species (i.e., a xenogenic cell population). The subject to be treated can be a human, non-human primate, a sheep, cow, dog, rat, or mouse. In some embodiments, the subject to be treated is a human patient.

In an exemplary, non-limiting, embodiment, muscle-derived stem cells are obtained as follows. Ewes are anesthetized via Ketamine/Valium (10 mg/kg and 0.25 mg/kg) (induction) and mechanically ventilated using inhaled isoflurane (2.0%) to maintain anesthesia through an endotracheal tube. A biopsy of the sternocleidomastoid muscle (approximately 3 cm by 1 cm and weighing 3 grams), or another suitable muscle described herein, is obtained via blunt dissection and excision with a No 15 scalpel blade and immediately transferred to Initial Myogenic Culture Medium ((IMCM); Ham's F-10 nutrient mixture culture medium; e.g., from Invitrogen; Cat#11550-043) supplemented with 20% Fetal Bovine Serum (HyClone Laboratories, Inc. South Logan, Utah 84321; Cat# SH30070.03), 1% Penicillin/Streptomycin/Amphotericin B (Cellgro; Mediatech, Inc Manassas, Va. 20109; Cat#30-004-CI), and 1% Chicken Embryo Extract (SeraLab; United Kingdom; Cat#CE-650-J) and kept on ice for twelve hours. Muscle tissue is then minced into small pieces and digested in 0.2% collagenase type I (Collagenase Type 1-Filtered; Worthington Biochemical Corporation, Lakewood, N.J.; Cat# LS004214) in IMCM in a shaking water bath at 37° C. for 2 hours. IMCM is added to digested muscle to terminate the reaction and individual fibers were dissociated by vigorous pipetting. Digested tissue is filtered through a strainer with a pore size of 300 μm and followed by three washes with IMCM by centrifugation at 1500 RPM for five minutes per wash. The pellet is resuspended in IMCM and muscle fibers transferred into three wells of a 0.1% gelatin (Gelatin from porcine skin, Type A; Sigma-Aldrich; Cat# G1890)-coated six-well plate. The presence of single muscle fibers is confirmed by microscopy and plates left in a 5% CO₂ incubator at 37° C. overnight. To prevent potential overgrowth of fibroblasts and other faster growing cells, after 24 hours the supernatant containing non-adhered muscle stem cells is transferred into three different wells of a 0.1% gelatin coated 6 well plate. Medium is changed at day 4 and 6. When the primary culture reaches 70% confluency, about 9 days after initial plating, it is trypsinized and split. Optionally, to track the MdSCs post injection into the tongue, sheep MdSCs are transduced with a viral vector to express a reporter protein (e.g., green fluorescent protein (GFP)). In some cases, the viral vector to be used is a lentivirus encoding EGFP. MdSCs are transduced with the EGFP-lentivirus at passage 2 in the presence of 8 μg/ml Protamine Sulfate (Sigma-Aldrich; Cat#p4020). After the second passage, growth media is switched to a Myogenic Culture Medium [(MCM); F-10 culture media; Gibco; Grand Island, N.Y.; Cat#11550-043) supplemented with 10% Fetal Bovine Serum (HyClone Laboratories, Inc. South Logan, Utah 84321; Cat# SH30070.03), 1% Penicillin/Streptomycin (HyClone Laboratories, Inc. South Logan, Utah 84321; Cat# J110381). MdSC cultures typically consist of satellite cells and myoblasts. Cultures are routinely passaged at 70% confluency to prevent myotube formation.

For transplantation into a subject suffering from dysphagia, the MdSC population can include from about 10⁴ MdSCs to about 10⁸ MdSCs, e.g., 10⁵ MdSCs, 10⁶ MdSCs, 10⁷ MdSCs, or another number of MdSCs from about 10⁴ MdSCs to about 10⁸ MdSCs. Typically the MdSCs are administered by injection into one or more loci within a muscle required for swallowing. Such muscles include, tongue muscle, pharynx muscle, palate muscle, and strap muscle. In some embodiments, the cells are administered at between one to about twenty injections sites with each site receiving an equal number of cells, e.g., 2, 3, 4, 5, 6, 7, 8, 9, 10, 12, 14, 16, 18 or another number of injection sites from one to about twenty injection sites. In some embodiments, MdSCs are administered to a combination of any of the above-muscles required for swallowing. In other embodiments, the cells are administered to multiple sites of the tongue muscle.

Muscle function, structure, and swallowing before and after treatment by the methods described herein can be assessed by any of a number of methods known in the art, including, but not limited to: lingual tensometry, α-Bungarotoxin and βIII Tubulin Staining, and myofiber cytometry to determine muscle thickness. In some embodiments, the method results in at least a 10% to about a 30% increase in lingual strength relative to lingual strength in the untreated subject, e.g., about a 12%, 14%, 15%, 20%, 25%, 27%, 28%, or another increase in lingual strength from at least 10% to about a 30% increase.

### EXAMPLES

The invention will be more fully understood upon consideration of the following non-limiting examples, which are offered for purposes of illustration, not limitation.

### Example 1

### Isolation, Culture, and Administration of MdSCs in a Large Animal Model of Dysphagia

Muscle Biopsy and Cell Culture Technique.

This study protocol was approved by the University of California Davis Animal Care and Use Committee and institutional guidelines. Two dorper cross ewes were anesthetized via Ketamine/Valium (10 mg/kg and 0.25 mg/kg) (induction) and mechanically ventilated using inhaled isoflurane (2.0%) to maintain anesthesia through an endotracheal tube. A biopsy of the sternocleidomastoid muscle (approximately 3 cm by 1 cm and weighing 3 grams) was obtained via blunt dissection and excision with a No 15 scalpel blade and immediately transferred to Initial Myogenic Culture Medium [F-10 culture media; Invitrogen; Cat#11550-043) supplemented with 20% Fetal Bovine Serum (HyClone Laboratories, Inc. South Logan, Utah 84321; Cat# SH30070.03), 1% Penicillin/Streptomycin/Amphotericin B (Cellgro; Mediatech, Inc Manassas, Va. 20109; Cat#30-004-CI), and 1% Chicken Embryo Extract (SeraLab; United Kingdom; Cat#CE-650-J) and kept on ice for twelve hours. Muscle tissue was then minced in small pieces and digested in 0.2% collagenase type I (Collagenase Type 1-Filtered; Worthington Biochemical Corporation, Lakewood, N.J. o8701; Cat#LS004214) in initial myogenic culture medium (IMCM) in a shaking water bath at 37° C. for 2 hours. IMCM was added to digested muscle to terminate the reaction and individual fibers were dissociated by rigorous pipetting. Digested tissue was filtered through a strainer with a pore size of 300 uM and followed with 3 washes with IMCM by centrifugation at 1500 rpm for 5 min per wash. The pellet was resuspended in IMCM and muscle fibers transferred into 3 wells of a 0.1% gelatin (Gelatin from porcine skin, Type A; Sigma-Aldrich; Cat# G1890) coated 6 well plate. Single fibers were confirmed by microscopy and plates left in a 5% CO₂ incubator at 37° C. overnight. To prevent potential overgrowth of fibroblasts and other faster growing cells, the supernatant containing non-adhered muscle stem cells was transferred into 3 different wells of a 0.1% gelatin coated 6 well plate after 24 hours. Medium was changed at day 4 and 6. When the primary culture reached 70% confluency after 9 days of initial plating, it was trypsinized and split. To track the MdSCs post injection into the tongue, sheep MdSCs were transduced with expressing green fluorescent protein (GFP) expressing lentivirus vector at passage 2 in the presence of 8 μg/ml Protamine Sulfate (Sigma-Aldrich; Cat#p4020). After the second passage, growth media was switched to a Myogenic Culture Medium [F-10 culture media; Gibco; Grand Island, N.Y.; Cat#11550-043) supplemented with 10% Fetal Bovine Serum (HyClone Laboratories, Inc. South Logan, Utah 84321; Cat# SH30070.03), 1% Penicillin/Streptomycin (HyClone Laboratories, Inc. South Logan, Utah 84321; Cat# J110381). MdSC cultures consisted of satellite cells and myoblasts, with cultures routinely passaged at 70% confluency to prevent myotube formation.

Bilateral Hypoglossal Nerve Stimulator Implantation and Partial Hypoglossal Nerve Denervation.

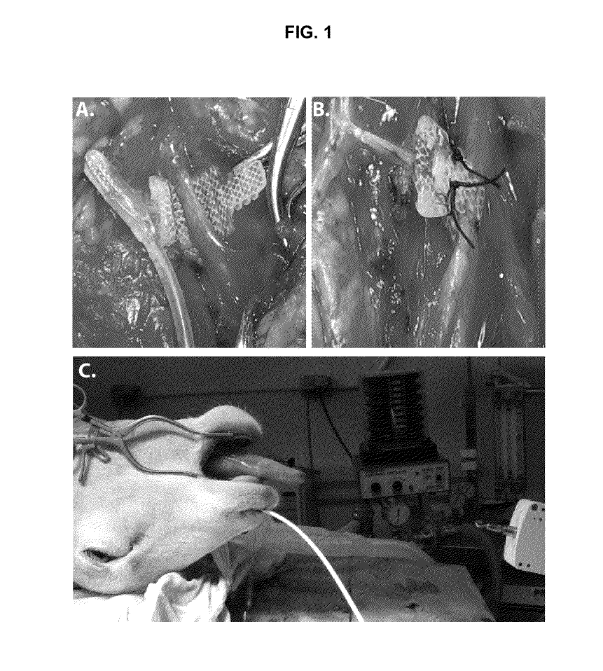

Three weeks following skeletal muscle biopsy procedures, animals were brought back to the operating room, anesthetized as described above, and secured in a supine position. The hypoglossal nerves (bilateral) were surgically exposed using sterile technique through a horizontal apron incision. To produce the partial denervation model, the hypoglossal nerves were bilaterally compressed with cuff electrodes. A half-cuff 6-ring electrode (MED-EL; Innsbruck, Austria) was placed on the main branch of both hypoglossal nerves (FIG. 1A, 1B). Electrodes were connected via multichannel leads to an implantable pulse stimulator (MED-EL, Innsbruck, Austria) capable of transcutaneous activation using an externally placed magnet. Stimulators were tunneled and secured in the subcutaneous tissue of the anterolateral neck and the wound closed in typical multilayer fashion.

Baseline Maximal Lingual Contractile Force and Tongue Biopsy.

Immediately following hypoglossal nerve stimulator placement, lingual tensometer testing, high-resolution manometry (HRM) lingual pressure testing and incisional tongue biopsies (approximately 5 g) were performed for baseline measures of tongue strength and muscle fiber histology respectively. For tensometer testing, a heavy-gauge monofilament suture (#0 Nylon, Ethicon) was placed at the anterior aspect of the tongue, looped back and fixed to a high-capacity steel hook (G1038; Mark-10 Corporation; Copiague, N.Y.) mounted on a digital force transducer (M4-100 force gauge; Mark-10 Corporation; Copiague, N.Y.) (FIG. 1C). The tongue was manually protruded from the mouth and the line tension adjusted to achieve a preload between 100 and 105 g. Bilateral simultaneous super-maximum nerve stimulation was used to elicit a net maximal lingual retrusive force, a concept previously demonstrated in humans by Eisele et al. (20). Using an externally placed magnet, the implanted electrodes were activated to initiate a train of ten one second supramaximal (3.5 mA) bursts. Line tension on the lingual suture was transduced by the force gauge and captured at 1000 Hz on a personal computer using data acquisition software (LabView; National Instruments; Austin, Tex.). HRM was performed simultaneously during hypoglossal nerve stimulation to assess maximal lingual pressure via a 36-channel 4.2 mm esophageal catheter and portable manometery workstation (MdSC-1286 catheter; ManoScan 360 monometer; Given Imaging; Yoqneam, Israel). Manometry data was acquired on an apple laptop computer using data acquisition software developed for this purpose (ManoView 2.0.1; Given Imaging; Yoqneam, Israel).

Autologous MdSC Injection.

Immediately following baseline testing, autologous MdSCs in PBS (Fisher-Scientific) were injected into the right and left side of the tongue of both animals. Each sheep received 5×10⁸ cells, delivered equally among five injections on each side of the tongue for a total of 10 injections per animal.

Post-MdSC Maximal Tongue Contractile Force Testing and Biopsy.

Two-months post-MdSC injection, lingual tensometer and HRM testing was again conducted as described above to evaluate tongue strength. Following testing, tongues were harvested for subsequent histological analysis and animals were euthanized while under anesthesia.

Detection of GFP (Green Fluorescent Protein) Marker by Immunohistochemistry.

Harvested tongue specimens were incubated in 4% paraformaldehyde in 4° C. for 16 hours, then transferred into 30% sucrose and incubated in 4° C. for 24 hours. Cryosectioning was performed at 12 μm thickness and sections were then hydrated by two changes of PBS. The slides were immersed in L.A.B epitope retrieval solution (Polysciences, Inc; Cat#24310) for 15 minutes and the endogenous peroxidase was blocked by incubation with 3% H₂O₂ (Sigma-Aldrich, Cat# H1009) in methanol for five minutes. To block non-specific binding sites, sections were incubated with 1.5% normal horse serum [Vectastain ABC kit (Rabbit IgG); Vector Laboratories, Inc, Cat# PK-6102] in a humidification chamber for 30 minutes. The sections were then incubated with rabbit polyclonal anti-GFP antibody (Cat# ab290) overnight at 4° C. at a 1:50 dilution. After washing with PBS three times, sections were incubated with anti-rabbit biotinylated secondary antibody for 30 minutes at room temperature. After washing with PBS two times, sections were incubated with Vectastain ABC kit (Rabbit IgG; Vector Laboratories, Inc, Cat# PK-6101] for 30 minutes at room temperature. Following washings with two changes of PBS, sections were incubated with peroxidase substrate solution (chromogenic substrate 3,3′ diaminobenzidine tetrahydrochloride; Sigma-Aldrich; Cat# D5905) for 1 minute. The sections were then counter-stained in Modified Harris hematoxylin (Richrd-Allan Scientific; Cat#72704) for one minute before dehydrating and mounting (Eukitt; Electron microscopy Science; Cat#15322).

Haemotoxylin and Eosin Staining

Sections were fixed in 4% paraformaldehyde and then incubated in 30% sucrose. They were then embedded in paraffin, sectioned (2 μm thickness) and subjected to Haemotoxylin and Eosin (H & E) staining as follow: sections were rehydrated for one minute in water followed by 1 minute of staining with Modified Harris Hematoxylin and one minute with 0.25% Eosin (Fischer Scientific; Cat# SE22-500D). Sections were dehydrated using alcohol, cleared using Xylenes and mounted by Eukitt, a xylenes-based media.

Bungarotoxin-βIII Tubulin Staining

Specimen fixation and cryosectioning were performed as described in the immunohistochemistry section above. Cryosections were washed in PBS 3 times. They were then incubated in 0.3 M glycine in PBS for 20 min. The sections were permeabilized in 0.1% Triton X-100 in PBS for 20 min. They were washed with PBS 3 times and then blocked with 4% goat serum for 1 hour. After an overnight incubation in 4° C. with rabbit polyclonal class III β-tubulin antibody (Covance, Richmond, Calif., USA, NO.PRB-435p) at 1:1000 dilution they were incubated with Alexa Fluor 488 conjugated goat anti-rabbit IgG secondary antibody (Invitrogen, Cat# A11034) for 1 hour. To visualize the motor endplates in the same sections, they were incubated with Alexa Fluor 594 conjugated α-bungarotoxin (Molecular Probes, Cat# B13423) at 1:100 dilution for 1 hour. Sections were observed with the Olympus Flowviewer 1000 mpe confocal/two-photon microscope.

Myofiber Thickness and GFP Expression

Tongue specimens were assessed under microscopy following anti-GFP staining, and images were captured for subsequent analysis of myofibers. Myofiber diameter and GFP intensity were determined using Image J software [National Institute of Health (NIH); Bethesda, Md.] and utilizing previously described methods of Halum et al (13). Mean myofiber diameter pre-MdSC injection, and GFP⁺ and GFP⁻ myofiber diameter post-MdSC injection were calculated for each animal.

Outcome Measures.

Muscle fiber diameter (overall, GFP+, and GFP−) was the primary histologic outcome measure. Maximal contractile tongue force (g) and maximal lingual pressure (mmHg) were functional measures of tongue strength. Due to the small sample size (n=2), descriptive statistics were calculated (means, standard deviations, mean percentage change).

### Results

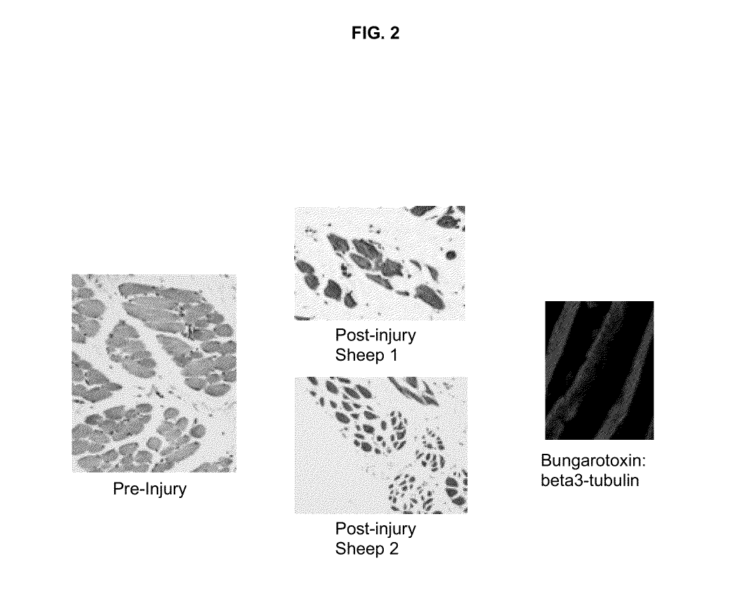

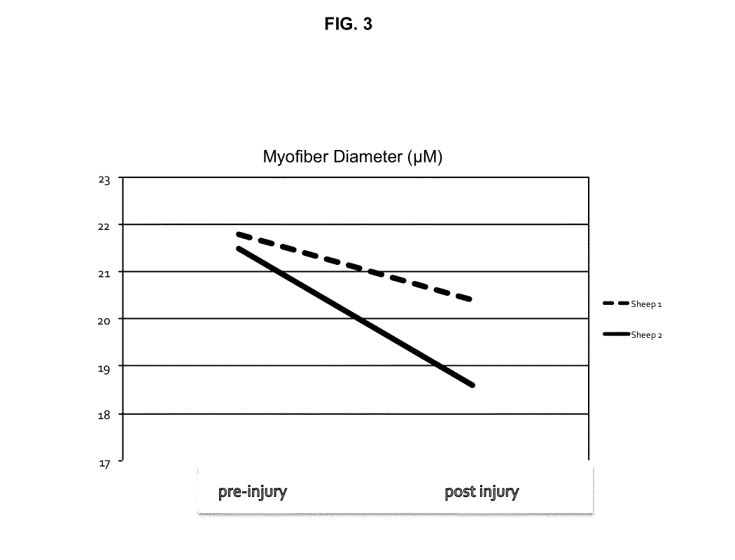

Partial nerve injury was induced in the sheep models of dysphagia using implantable electrodes placed on bilateral CN XII one month prior to implantation with muscle stem cells. The sheep were placed under anesthesia, implanted with electrodes in bilateral cranial nerve XII, underwent pre-muscle stem cell strength/manometry testing and tongue biopsy for baseline histology. FIG. 1 shows the hypoglossal nerve cuff electrode placement (FIG. 1A, B) and animal positioning for tongue tensometer and high resolution manometry testing (FIG. 1C). FIG. 2 shows the histological staining of the two sheep tongue pre- and post-partial nerve injury, showing the loss of muscle tissue in the tongue following injury. FIG. 3 and table 2 show the reduction in myofiber diameter following partial nerve injury in the two different mice.

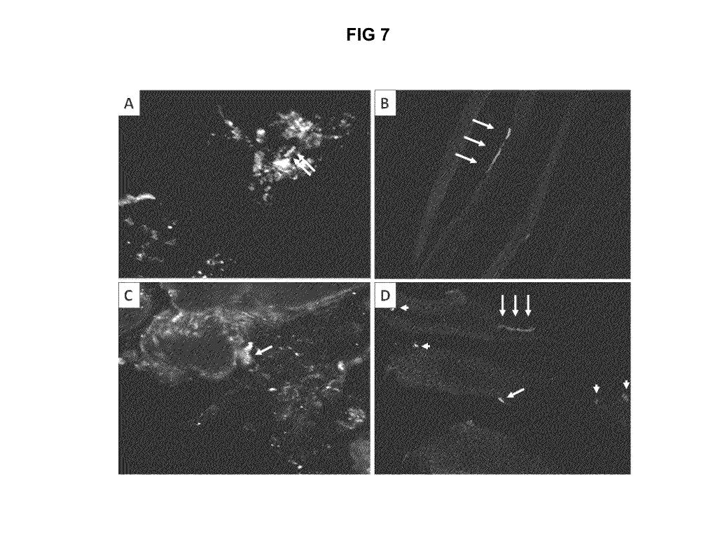

Hematoxylin and Eosin staining confirmed that a partial denervation injury had occurred in each animal as indicated by grouping, angulation, increased variability and atrophy of muscle fibers in both tongues. On average, myofiber diameter decreased by 8.18% and 12.07% in sheep 1 and 2 respectively (Table 1) and bungarotoxin-beta3 tubulin staining demonstrated reduced motor end-plate to nerve contact post-injury (FIG. 7), confirming a partial denervation injury. FIG. 7 depicts bungarotoxin-βIII tubulin stains of tongue specimens (at 12 μm section thickness). FIG. 7A (sheep 1) and FIG. 7C (sheep 2) represent motor endplate and neuronal staining at baseline before nerve injury with motor endplates (depicted with long arrows) demonstrating contact with the nerves in the area. At two months post-denervation and MdSC injection [B=sheep 1 and D=sheep 2], there was a reduction in neuronal (arrowheads) to motor end plate (long arrows) contact in both animals. Long arrows=motor endplates, and arrowheads=βIII tubulin positive nerve fibers not in direct contact/proximity with the motor endplates

In both animals, the pretreatment control tongue specimens consistently demonstrated from 93-100% of motor endplates to have nerve contact FIG. 7 (A, B).

TABLE 2
Mean (SD) muscle fiber diameter pre vs. post autologous MSC
injection indicating a reduction in muscle fiber diameter in both
animals and confirming a partial denervation tongue injury.

| | Pre MSC | Post MSC | Mean Change |
|---|---|---|---|
| Sheep 1 | 22.24 (6.34) | 20.42 (7.47) | −8.18% |
| Sheep 2 | 21.20 (6.92) | 18.64 (8.80) | −12.07% |

Following the nerve injury, sheep 1 tongue specimen demonstrated an average of 47.3% (±9.8%) of motor endplates with nerve contact, while sheep 2 tongue demonstrated an average of 37.0% (±13.6%) motor endplates with nerve contact.

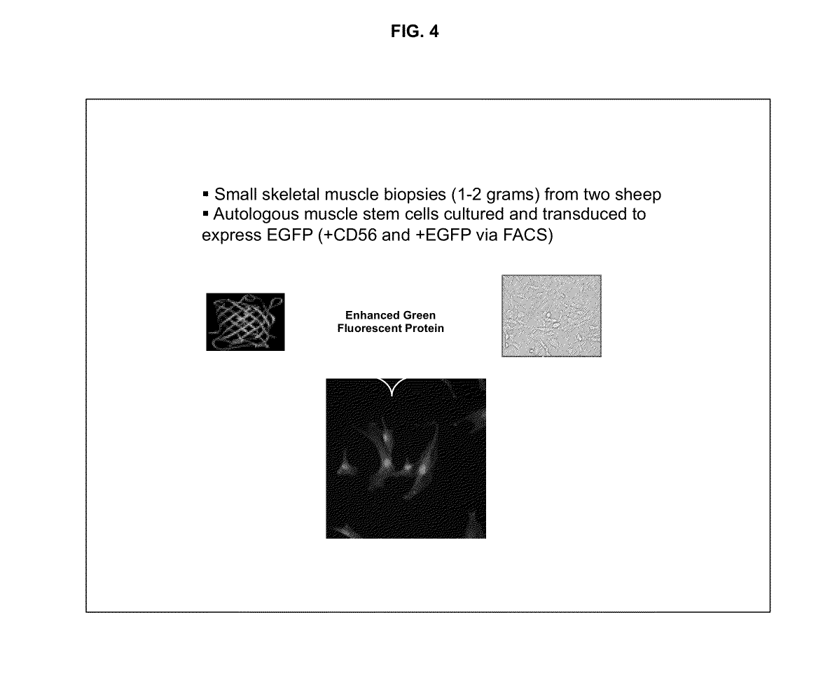

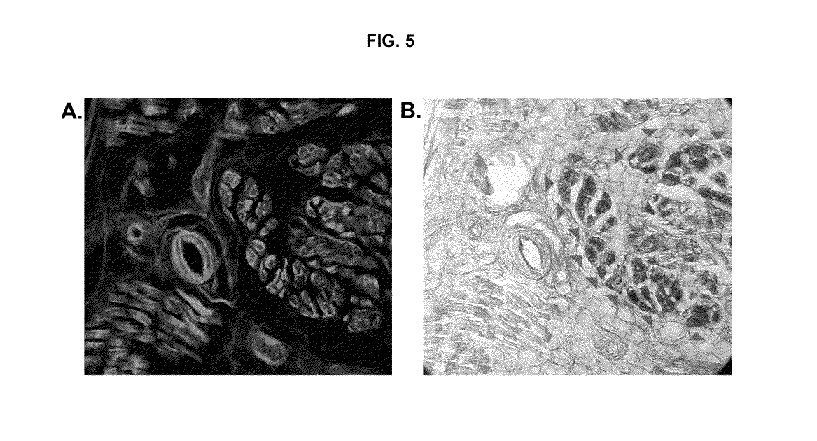

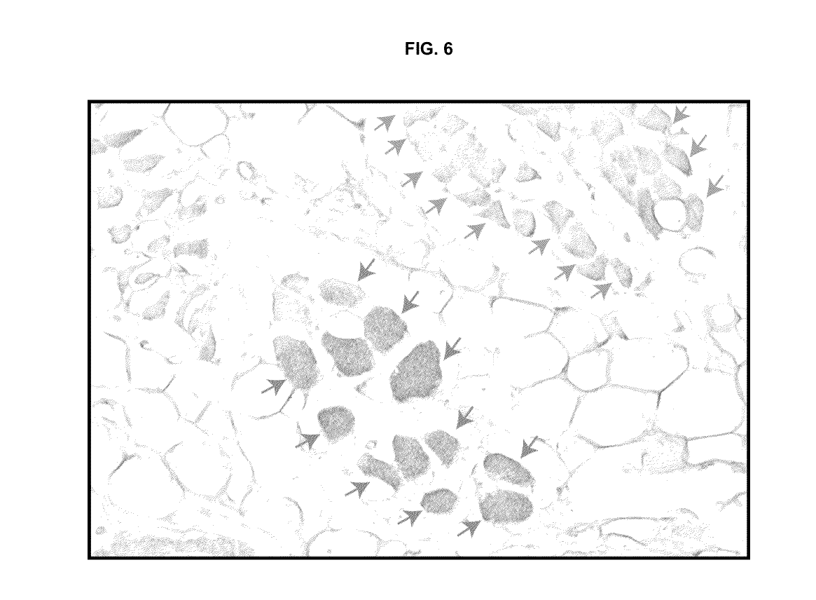

One month following induction of partial nerve injury, sheep were given diffuse injection of at least 10⁶ GFP positive muscle stem cells into the tongue. The GFP-positive muscle stem cells were obtained by small skeletal muscle biopsies (about 1-2 grams) from the sheep and transduced for EGFP expression and EGFP⁺ cells were isolated using fluorescence activated cell sorting (FACS) for CD56⁺ and EGFP⁺ (FIG. 4). The presence of implanted muscle stem cells was observed in tongue samples by fluorescence microscopy to detect EGFP fluorescence and by immunohistochemistry with an anti-EGFP antibody (FIG. 5 and FIG. 6).

FIG. 5 depicts cross sectional tongue muscle fiber specimen (12 μm section thickness) from Sheep 1 imaged at ×20 magnification in fluorescent (A) and brightfield (B) modes indicating the presence of GFP⁺ myofibers (arrows in B) and the presence of fused MdSC at two-months post injection in certain areas of the tongue. Note that the artifact throughout the fluorescent section (A) precluded accurate identification of GFP⁺ myofibers based on fluorescence alone, and therefore, anti-GFP staining with immunohistochemistry was performed to detect GFP⁺ myofibers with high specificity (see FIG. 6).

FIG. 6 depicts detection of GFP (Green Fluorescent Protein) marker by immunohistochemistry with GFP⁺ myofibers (darker arrows, middle bottom) representing the areas where injected GFP⁺ muscle stem cells have fused with innate myofibers; note that GFP⁺ myofibers appear larger in diameter than GFP⁻ (lighter arrows, top, top-right) myofibers.

MdSC Survival and Increased Myofiber Diameter in EGFP⁺ Fibers.

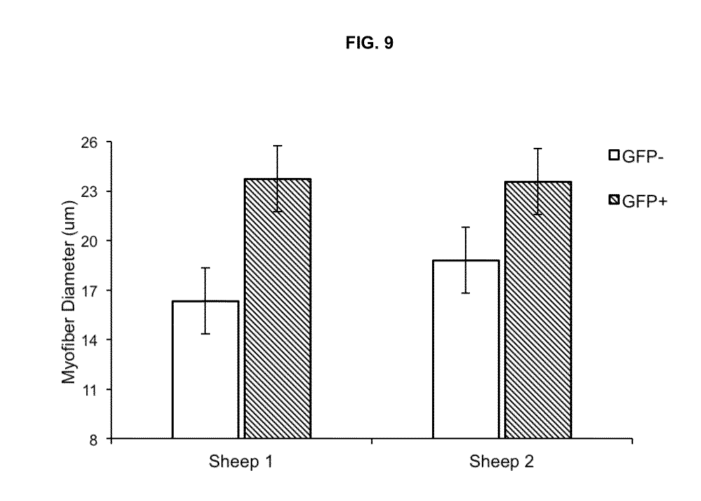

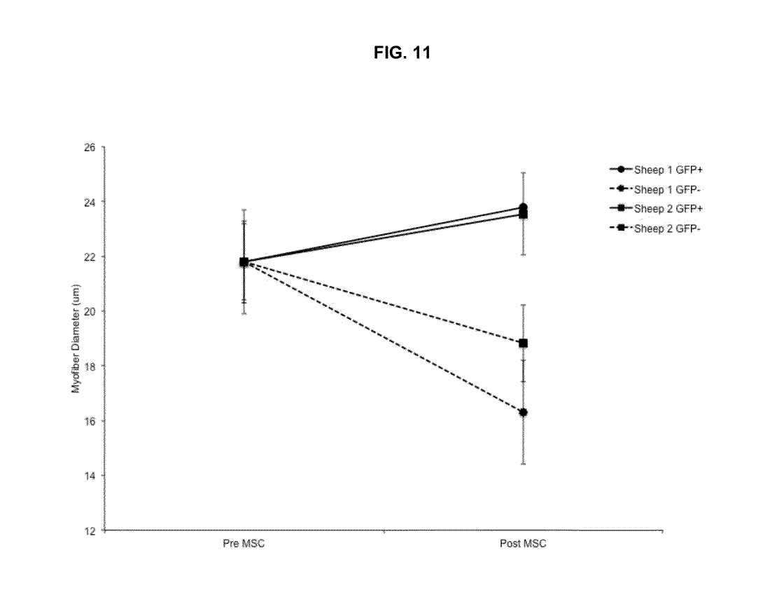

The presence of GFP⁺ myofibers was confirmed by immunofluorescence microscopy (FIG. 5A), and indicated that MdSCs survived and fused into myotubes in specific areas of the tongue. Analysis with Image J software revealed high fluorescence intensity in all slides. To discern the GFP⁺ areas from artifact, immunohistochemistry with primary anti-GFP was performed and slides analyzed by brightfield microscopy (FIG. 5B). Mean muscle fiber diameter for GFP+ muscle fibers were 45.44% and 25.08% larger than GFP− muscle fiber diameters in animals 1 and 2, respectively, status post MdSC injection (FIG. 6, FIG. 5). Due to the partial denervation injury, mean muscle fiber diameter decreased pre vs. post MdSC injection in GFP⁻ muscle fibers. However in GFP⁺ fibers, mean muscle fiber diameter increased suggesting that the MdSCs had not only attenuated atrophy, but had enlarged the myofibers that they had fused with, resulting in increased diameters in the GFP⁺ myofibers compared to the nondenervated state (FIG. 9, FIG. 11).

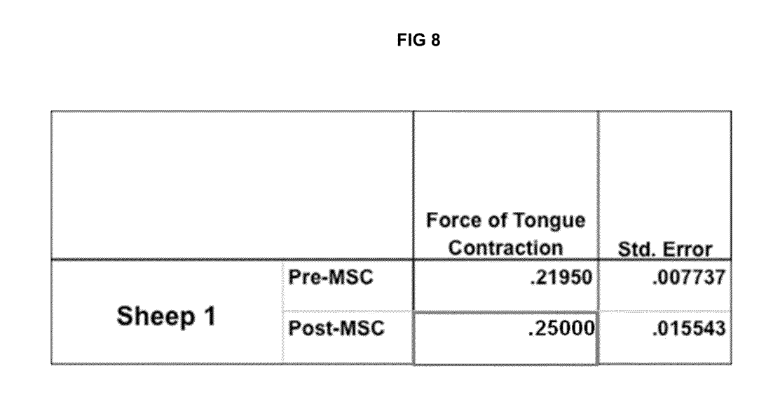

Two months following the initial biopsy (one month following muscle stem cell implantation), a post-muscle stem cell implantation tongue strength/manometry testing is done and the tongues are harvested for analysis. In Sheep 1, there was an increase in force of muscle contraction post muscle stem cell implant compared to pre muscle stem cell implantation (FIG. 8). Myofibers that were GFP⁺ showed increased diameter compared to GFP− myofibers (FIG. 9). This shows that GFP positive muscle stem cells could increase the size of myofibers in the tongue following implantation. FIG. 11 showed comparison of myofiber size from two individual sheep in muscle that are GFP⁺ versus those that are GFP⁻. This shows that implantation of GFP positive muscle stem cells could increase the size of tongue muscle in these animal models.

FIG. 9 depicts mean muscle fiber diameter for GFP⁺ (striped bars) are larger than GFP⁻ (white bars) muscle fibers in both animals post-MdSC, showing attenuation of muscle fiber atrophy where MdSCs fused.

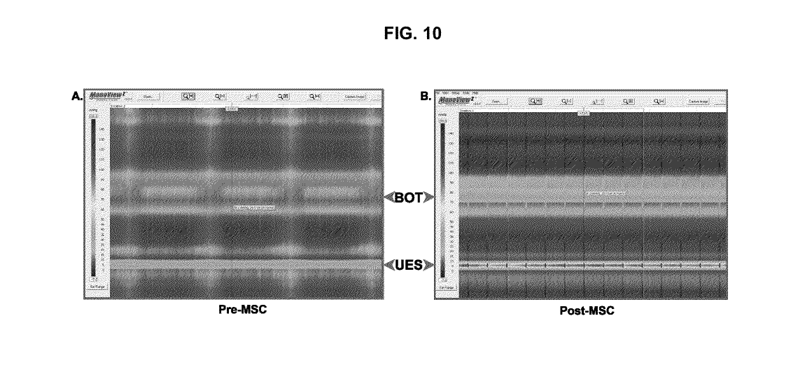

FIG. 10 depicts representative High Resolution Manometery output from Sheep 1 Pre-MdSC (A) and 2 months post-MdSC injection (B), indicating an increase in base of tongue (BOT) and upper esophageal sphincter (UES) pressure two-months post-MdSC injection (B).

FIG. 11 depicts mean muscle fiber diameter for GFP+ myofibers increased across time points while GFP− myofiber diameter decrease indicating formation of new muscle fibers and atrophy of myofibers post-denervation injury respectively.

Maximal tongue contractile force increased post MdSC injection in Sheep 1. Mean maximal contraction was 219 g (7.70) and 300 g (15.10) for pre- and post-MdSC injection time points respectively, indicating a 27% lingual strength increase from pre-injury levels. Mean pharyngeal pressure at the level of the base of tongue was 16.2 mmHg at baseline and increased to 30.3 mmHg post-MdSC treatment, indicating a 55% increase in pharyngeal pressure generation post-MdSC injection in Sheep 1 (FIG. 8). The partial denervation injury precluded post-MdSC simulation in Sheep 2. The hypoglossal nerves could not be stimulated at the two-month time post injection time point.

This example shows that autologous MSCs fused with partially denervated tongue myofibers and that post muscle stem cell injection tongue had increased beyond the pre-injury baseline in select cases. The injected muscle stem cells attenuated the tongue myofiber atrophy seen with denervation injury, with those myofibers that were GFP⁺ and had fused with muscle stem cells had myofiber diameter greater to non-injured control diameters.

Models of laryngeal denervation in rat and cardiac ischemia in humans support the viability of muscle stem cell injection attenuating muscle atrophy and improving the force of contraction. Dib et. al. (Circulation. 2005 Sep. 20; 112:1748-55) showed safety and feasibility of autologous myoblast transplantation in patients with ischemic cardiomyopathy with a four year follow up. Halum et. al. (Laryngoscope 2007, 117:917-22 and Laryngoscope 2008; 118:1308-12) showed muscle stem cell survival following laryngeal transplant after recurrent laryngeal nerve injury.

This study is the first investigation evaluating the use of autologous MdSC therapy for treatment of the partially denervated tongue in a large animal model. Results showed that MdSCs survived for at least a two-month period, and MdSC fusion with partially denervated myofibers enlarged the myofibers beyond their baseline pre-denervation state. Additionally, the findings show that these cellular myogenic changes may lead to functional gains in tongue contractile force and base of tongue pressure.

The histologic findings of larger myofiber diameters in GFP⁺ myofibers shows that MDSCs that fuse in the partially denervated ovine tongue have the ability to attenuate muscle atrophy. Similar effects have been noted in the rodent denervated thyroarytenoid muscle (12, 13, 16). The additional finding that muscle fiber size increased beyond pre-injury levels may occur secondary to the MDSCs fusing with existing myofibers or the MDSCs self-fusing to form new myofibers.

The present disclosure finds utility in the restoration of the weakened and denervated tongue following neurologic or surgical insult. To our knowledge, this is the first disclosure of the impact of MdSC therapy on tongue function. The observed 27% increase in maximal tongue contractile force and 55% increase in lingual pressure pre vs. post MdSC injection in the presence of a partial denervation tongue injury showed that the noted muscle fiber changes/growth may lead to functional gains in tongue strength in one animal. This finding has clinical significance because the weakened tongue contributes to impairments in swallowing that include: poor bolus manipulation and transfer, increased oral stasis, decreased bolus pressure generation and resultant pharyngeal residue that result in aspiration.

The present invention has been described in connection with what are presently considered to be the most practical and preferred embodiments. However, the invention has been presented by way of illustration and is not intended to be limited to the disclosed embodiments. Accordingly, one of skill in the art will realize that the invention is intended to encompass all modifications and alternative arrangements within the spirit and scope of the invention as set forth in the appended claims.

### REFERENCES

1. Bello-Haas V D, Florence J M, Kloos ADet al. A randomized controlled trial of resistance exercise in individuals with ALS. Neurology 2007; 68:2003-2007.
2. Dalbello-Haas V, Florence J M, Krivickas L S. Therapeutic exercise for people with amyotrophic lateral sclerosis or motor neuron disease. Cochrane Database Syst Rev 2008:CD005229.
3. McCrate M E, Kaspar B K. Physical activity and neuroprotection in amyotrophic lateral sclerosis. Neuromolecular medicine 2008; 10:108-117.
4. Nguyen N P, Moltz C C, Frank Cet al. Dysphagia following chemoradiation for locally advanced head and neck cancer. Annals of oncology: official journal of the European Society for Medical Oncology/ESMO 2004; 15:383-388.
5. Agarwal J, Palwe V, Dutta Det al. Objective assessment of swallowing function after definitive concurrent (chemo)radiotherapy in patients with head and neck cancer. Dysphagia 2011; 26:399-406.
6. Falsetti P, Acciai C, Palilla Ret al. Oropharyngeal dysphagia after stroke: incidence, diagnosis, and clinical predictors in patients admitted to a neurorehabilitation unit. Journal of stroke and cerebrovascular diseases: the official journal of National Stroke Association 2009; 18:329-335.
7. Kidd D, Lawson J, Nesbitt R, MacMahon J. Aspiration in acute stroke: a clinical study with videofluoroscopy. The Quarterly journal of medicine 1993; 86:825-829.
8. Drory V E, Goltsman E, Reznik J G, Mosek A, Korczyn A D. The value of muscle exercise in patients with amyotrophic lateral sclerosis. Journal of the neurological sciences 2001; 191:133-137.
9. Kaspar B K, Frost L M, Christian L, Umapathi P, Gage F H. Synergy of insulin-like growth factor-1 and exercise in amyotrophic lateral sclerosis. Annals of neurology 2005; 57:649-655.
10. Kirkinezos I G, Hernandez D, Bradley W G, Moraes C T. Regular exercise is beneficial to a mouse model of amyotrophic lateral sclerosis. Annals of neurology 2003; 53:804-807.
11. Plowman-Prine E K, Sapienza C M, Okun MSet al. The relationship between quality of life and swallowing in Parkinson's disease. Movement disorders: official journal of the Movement Disorder Society 2009; 24:1352-1358.
12. Halum S L, Hiatt K K, Naidu M, Sufyan A S, Clapp D W. Optimization of autologous muscle stem cell survival in the denervated hemilarynx. The Laryngoscope 2008; 118:1308-1312.
13. Halum S L, Naidu M, Delo D M, Atala A, Hingtgen C M. Injection of autologous muscle stem cells (myoblasts) for the treatment of vocal fold paralysis: a pilot study. The Laryngoscope 2007; 117:917-922.
14. Dib N, Michler R E, Pagani FDet al. Safety and feasibility of autologous myoblast transplantation in patients with ischemic cardiomyopathy: four-year follow-up. Circulation 2005; 112:1748-1755.
15. Gavira J J, Herreros J, Perez Aet al. Autologous skeletal myoblast transplantation in patients with nonacute myocardial infarction: 1-year follow-up. The Journal of thoracic and cardiovascular surgery 2006; 131:799-804.
16. Halum S L, McRae B, Bijangi-Vishehsaraei K, Hiatt K. Neurotrophic factor-secreting autologous muscle stem cell therapy for the treatment of laryngeal denervation injury. The Laryngoscope 2012; 122:2482-2496.
17. Bunaprasert T, Hadlock T, Marler Jet al. Tissue engineered muscle implantation for tongue reconstruction: a preliminary report. The Laryngoscope 2003; 113:1792-1797
18. Luxameechanporn T, Hadlock T, Shyu J, Cowan D, Faquin W, Varvares M. Successful myoblast transplantation in rat tongue reconstruction. Head & neck 2006; 28:517-524.
19. Sasao N, Hirayama E, Kim J. Characterization of heterokaryons between skeletal myoblasts and preadipocytes: myogenic potential of 3T3-L1 preadipocytes. European journal of cell biology 2003; 82:97-103.
20. Eisele D W, Smith P L, Alam D S, Schwartz A R. Direct hypoglossal nerve stimulation in obstructive sleep apnea. Archives of otolaryngology—head & neck surgery 1997; 123:57-61.
21. Luxameechanporn T, Blair C, Kirtsreesakul V, Thompson K, Naclerio R M. The effect of treatment with moxifloxacin or azithromycin on acute bacterial rhinosinusitis in mice. International journal of infectious diseases: IJID: official publication of the International Society for Infectious Diseases 2006; 10:401-406.

The invention claimed is:

1. A method for treating dysphagia, comprising administering to a subject in need thereof a therapeutically effective dose of an isolated muscle-derived stem cell population into a muscle used for swallowing.

2. The method of claim 1, wherein the muscle used for swallowing is selected from the group consisting of a tongue muscle, a pharynx muscle, a palate muscle, and a strap muscle.

3. The method of claim 1, wherein the isolated muscle-derived stem cell population is administered to the subject in the tongue muscle.

4. The method of claim 1, wherein the isolated muscle-derived stem cell population is administered to the subject in the pharynx muscle.

5. The method of claim 1, wherein the isolated muscle-derived stem cell population is administered to the subject in the palate muscle.

6. The method of claim 1, wherein the isolated muscle-derived stem cell population is administered to the subject in the strap muscle.

7. The method of claim 1, wherein at least about 10⁴ muscle-derived stem cells are administered.

8. The method of claim 1, wherein at least about 10⁵ Muscle-derived stem cells are administered.

9. The method of claim 1, wherein at least about 10⁶ Muscle-derived stem cells are administered.

10. The method of claim 1, wherein at least about 10⁷ stem cells are administered.

11. The method of claim 1, wherein at least about 10⁸ stem cells are administered.

12. The method of claim 1, wherein the muscle-derived stem cell population is obtained from sternocleidomastoid muscle.

13. The method of claim 1, wherein the muscle-derived stem cell population is autogenic, allogenic, or xenogenic.

14. The method of claim 13, wherein the muscle-derived stem cell population is autogenic.

15. The method of claim 1, wherein the dysphagia is neurogenic dysphagia.

16. The method of claim 1, wherein the subject is human.

17. The method of claim 1, further obtaining and culturing the isolated muscle-derived stem cell population prior to the administration.

18. The method of claim 1, wherein lingual strength in the subject following the treatment is at least 10% greater than lingual strength in the subject prior to the treatment.

19. The method of claim 18, wherein the lingual strength is at least 20% greater.

20. The method of claim 18, wherein the lingual strength is at least 25% greater.
